(19) United States
(12) Patent Application Publication
(10) Pub. No.: US 2003/0073662 A1
(43) Pub. Date: Apr. 17, 2003

(54) Regulation of cellular functions by ectopic expression of non-endogenous cell signalling receptors

(75) Inventors: Marvin C. Gershengorn, New York, NY (US); Erik S. Falck-Pedersen, Dobbs Ferry, NY (US); Ronald G. Crystal, New York, NY (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Cornell Research Foundation, Inc., Ithaca, NY

(21) Appl. No.: 10/291,007

(22) Filed: Nov. 8, 2002

Related U.S. Application Data
(63) Continuation of application No. 09/011,624, filed on Jun. 4, 1998, now abandoned.
Application No. 09/011,624 is a 371 of international application No. PCT/US96/13077, filed on Aug. 12, 1996.
(60) Provisional application No. 60/002,254, filed on Aug. 14, 1995.

(51) Int. Cl. A61K 48/00; C07K 14/705; C12N 15/867

(52) U.S. Cl. 514/44; 424/93.21; 435/456; 530/350

(57) ABSTRACT

The present invention is directed to an in vivo cell transformed with DNA encoding a cell signalling receptor not endogenous to the cell. The cell signalling receptor is capable of activating a signal transduction pathway endogenous to the cell, and the cell signalling receptor can be controllably activated thereby controllably activating the signal transduction pathway so as to regulate a cell function controlled by the signal transduction pathway. The invention also provides a method of ectopically expressing a non-endogenous receptor in a cell, and a method of regulating a cell function in vivo. The method of regulating a cell function comprises transforming a cell with DNA encoding a cell signalling receptor not endogenous to the cell, as above, and controllably exposing the cell to an extracellular molecule capable of activating the foreign cell signalling receptor. Activation of the cell signalling receptor activates the endogenous signal transduction pathway so as to regulate a cell function controlled by the endogenous signal transduction pathway.

### CROSS-REFERENCE TO RELATED PATENT APPLICATIONS

This patent application is a continuation of copending U.S. patent application Ser. No. 09/011,624, filed Jun. 4, 1998, which is the national phase of International Application No. PCT/US96/13077, filed Aug. 12, 1996, which designates the United States and which claims the benefit of U.S. Provisional Patent Application No. 60/002,254, filed Aug. 14, 1995.

### STATEMENT REGARDING FEDERALLY SPONSORED RESEARCH AND DEVELOPMENT

This invention was made in part with Government support under Grant Numbers RO1 DK43036 awarded by the National Institutes of Health. The Government may have certain rights in this invention.

### FIELD OF THE INVENTION

The present invention relates to expression of a non-endogenous cell signalling receptor in a cell in order to gain control over a cellular function, and more particularly to the transformation of a cell with DNA encoding a non-endogenous cell signalling receptor that utilizes an endogenous signal transduction pathway in the cell, where the receptor can be controllably activated to regulate a cell function controlled by the signal transduction pathway.

### BACKGROUND OF THE INVENTION

Throughout this application various publications are referenced, many in parenthesis. Full citations for these publications are provided at the end of the Detailed Description. The disclosures of these publications in their entireties are hereby incorporated by reference in this application.

The differentiated function of a cell is defined, in part, by how it responds to its environment. While any given cell may be exposed to a myriad of signals, the specificity of cellular responses to extracellular regulatory molecules is modulated by the array of receptors present in that cell (1-5). Interaction of an extracellular regulatory molecule with its cell signalling receptor leads to activation of one or more intracellular signal transduction pathways, eventuating in a response(s) specific for the cell. The specificity of the cellular response is dictated by the specific binding characteristics of the receptor and the specificity of the molecular targets downstream of the signal transduction cascade. In the same cell, a number of different receptors may use the identical signal transduction pathway and thus activation of different receptors can sometimes elicit similar responses (1-5).

There are a number of inherited and acquired human disorders in which the disease phenotype can be corrected, or at least alleviated, if a particular signal transduction pathway could be activated in a specific cell/tissue in a controlled or regulated fashion. A general application of this approach would be activation of a signalling cascade to inhibit a process that has become overly stimulated in disease or to stimulate a cellular response that had been inhibited by disease. In these cases, the pathogenesis of the disease does not involve the specific signalling pathway. The disease phenotype would be overcome by other cellular responses triggered by a non-endogenous receptor. A specific application of this approach is in disorders in which there is dysregulation of differentiated functions secondary to abnormalities associated with extracellular regulatory molecules or their specific receptors. In these cases, the non-endogenous receptor could be employed to trigger the signalling pathway that had been disrupted by the disease.

### SUMMARY OF THE INVENTION

To this end, it is an object of the subject invention to provide a method to regulate the function of a diseased or normal cell/tissue so as to produce a response in the cell/tissue that would overcome or alleviate a diseased phenotype in that or in another cell/tissue.

More particularly, the invention provides an in vivo cell transformed with DNA encoding a cell signalling receptor not endogenous to the cell. The cell signalling receptor is capable of activating a signal transduction pathway endogenous to the cell, and the cell signalling receptor can be controllably activated thereby controllably activating the signal transduction pathway so as to regulate a cell function controlled by the signal transduction pathway.

The invention also provides a method of ectopically expressing a non-endogenous receptor in a cell. The method comprises selecting a cell for transformation with DNA encoding a cell signalling receptor not endogenous to the cell. The cell signalling receptor is capable of activating a signal transduction pathway endogenous to the cell. The cell is then transformed with DNA encoding the cell signalling receptor, and the DNA is expressed, thereby ectopically expressing the cell signalling receptor in the cell.

The invention further provides a method of regulating a cell function in vivo. The method comprises selecting a cell for transformation with DNA encoding a cell signalling receptor not endogenous to the cell. The cell signalling receptor is capable of activating a signal transduction pathway endogenous to the cell. The cell is then transformed with DNA encoding the cell signalling receptor, and the DNA is expressed, thereby ectopically expressing the cell signalling receptor in the cell. The in vivo cell is then controllably exposed to an extracellular molecule capable of activating the non-endogenous cell signalling receptor, wherein activation of the cell signalling receptor activates the endogenous signal transduction pathway so as to regulate a cell function controlled by the endogenous signal transduction pathway.

The invention thus provides a method for controlling a cellular function using extracellular molecules which do not normally control the particular cellular function. Various malfunctions of cellular functions due to desensitization of an endogenous cellular receptor, or lack of or mutation of an endogenous cellular receptor, can thereby be overcome using the compositions and methods of the subject invention.

### BRIEF DESCRIPTION OF THE DRAWINGS

These and other features and advantages of this invention will be evident from the following detailed description of preferred embodiments when read in conjunction with the accompanying drawings in which:

FIG. 1 is a Northern analysis of RNA from in vitro primary hepatocytes after incubation with adenovirus vectors, using a mouse TRH-R cDNA probe (lanes 1-6) or a human γ-actin cDNA probe (lanes 7-12);

FIG. 2 illustrates the binding of methylthyrotropin releasing hormone to hepatocytes following adenovirus-mediated in vitro transfer of the TRH-R cDNA;

FIG. 3 illustrates the TRH stimulation of inositol phosphate formation in hepatocytes following adenovirus-mediated in vitro transfer of the TRH-R cDNA;

FIG. 4 is a Northern analysis of liver RNA with a mouse TRH-R cDNA probe after the animal received the AdCMV.βgal vector (lane 1) or the AdCMV.mTRH-R vector (lane 2) in vivo;

FIG. 5 is a Northern analysis of liver RNA with a W-actin cDNA probe after the animal received the AdCMV.αgal vector (lane 3) or the AdCMV.mTRH-R vector (lane 4) in vivo;

FIG. 6 illustrates the binding of methylthyrotropin releasing hormone to primary hepatocytes derived from rats following adenovirus-mediated transfer of the TRH-R cDNA in vivo;

FIG. 7 illustrates the TRH stimulation of inositol phosphate formation in hepatocytes derived from rats following adenovirus-mediated transfer of the TRH-R cDNA in vivo;

FIG. 8 illustrates the modulation of serum glucose by TRH; and

FIG. 9 illustrates the average change in serum glucose levels.

### DETAILED DESCRIPTION OF THE INVENTION

The subject invention provides a recombinant in vivo cell comprising an in vivo cell transformed with DNA encoding a cell signalling receptor not endogenous to the cell. The cell signalling receptor is capable of activating a signal transduction pathway endogenous to the cell, and the cell signalling receptor can be controllably activated thereby controllably activating the signal transduction pathway so as to regulate a cell function controlled by the signal transduction pathway.

The invention also provides a method of ectopically expressing a non-endogenous receptor in a cell. The method comprises selecting a cell for transformation with DNA encoding a cell signalling receptor not endogenous to the cell. The cell signalling receptor is capable of activating a signal transduction pathway endogenous to the cell. The cell is then transformed with DNA encoding the cell signalling receptor, and the DNA is expressed, thereby ectopically expressing the cell signalling receptor in the cell.

The invention further provides a method of regulating a cell function. The method comprises selecting a cell for transformation with DNA encoding a cell signalling receptor not endogenous to the cell. The cell signalling receptor is capable of activating a signal transduction pathway endogenous to the cell. The cell is then transformed with DNA encoding the cell signalling receptor, and the DNA is expressed, thereby ectopically expressing the cell signalling receptor in the cell. The cell is then controllably exposed to an extracellular molecule capable of activating the cell signalling receptor, wherein activation of the cell signalling receptor activates the endogenous signal transduction pathway so as to regulate a cell function normally controlled by the endogenous signal transduction pathway.

The in vivo cell to be transformed can be any suitable cell in which control over a cellular function is desired. The control is accomplished by transforming the cell with DNA encoding a cell signalling receptor not endogenous to the cell, and allowing the non-endogenous receptor to utilize an endogenous signal transduction pathway of the cell. Cellular functions influenced by the signal transduction pathway can thereby be controlled by controlling the activation of the non-endogenous receptor (i.e., by controlled exposure of the non endogenous receptor to its extracellular signalling molecule).

The subject invention is best understood through a discussion of the various receptors and signal transduction pathways which can be utilized in accordance with the invention. Cells in higher animals normally communicate by means of hundreds of kinds of extracellular signalling molecules, including proteins, small peptides, amino acids, nucleotides, steroids, retinoids, fatty acid derivatives, and even dissolved gases such as nitric oxide and carbon monoxide. These signalling molecules relay a “signal” to another cell (a “target cell”), generally affecting a cellular function. In accordance with the subject invention, such extracellular signalling molecules are controllably administered or provided to a cell in vivo. The cell in vivo has been transformed so that the cell recognizes the extracellular signalling molecule via a non-endogenous receptor expressed by the cell. The non-endogenous receptor is specific for and binds the extracellular signalling molecule, and the presence of the non-endogenous receptor allows the in vivo cell to recognize the extracellular signalling molecule that it would not have recognized but for the presence of the non-endogenous receptor. The binding of the non-endogenous receptor to the extracellular signalling molecule then initiates a response in the transformed cell. As used herein, these receptors for extracellular signalling molecules are collectively referred to as “cell signalling receptors.”

Many cell signalling receptors are transmembrane proteins on the transformed cell surface; when they bind an extracellular signalling molecule (a ligand), they become activated so as to generate a cascade of intracellular signals that alter the behavior of the cell. As used herein, these receptors are collectively referred to as “cell surface signalling receptors”. In some cases, the receptors are inside the transformed cell and the signalling ligand has to enter the cell to activate them; these signalling molecules therefore must be sufficiently small and hydrophobic to diffuse across the plasma membrane of the cell. As used herein, these receptors are collectively referred to as “intracellular cell signalling receptors.”

Cell surface signalling receptors generally include three classes, defined by the transduction mechanism used. These are ion channel linked receptors, guanine nucleotide binding protein linked receptors, and enzyme linked receptors.

Ion channel linked receptors, also known as transmitter-gated ion channels, are involved in rapid synaptic signalling between electrically excitable cells. This type is signalling is mediated by a small number of extracellular signalling molecules known as neurotransmitters that transiently open or close the ion channel formed by the receptor protein to which they bind. The ion channel linked receptors belong to a family of homologous, multipass transmembrane proteins, and include ion channels for sodium, potassium, calcium, and chloride ions.

Ion channels are not continuously open. Instead they have “gates,” which open briefly and then close again. In most cases the gates open in response to a specific stimulus, such as the binding of a ligand (ligand-gated channels). The ligand can be either an extracellular signalling molecule, specifically a neurotransmitter (transmitter-gated channels), or an intracellular molecule, such as an ion (ion-gated channels) or a nucleotide (nucleotide-gated channels).

The second class of cell surface receptor proteins is the guanine nucleotide binding protein linked receptors. These receptors are also known as G protein linked receptors. These receptors act indirectly to regulate the activity of a separate plasma-membrane-bound target protein, which can be an enzyme or an ion channel. The interaction between the receptor and the target protein is mediated by a third protein, called a trimeric GTP-binding regulatory protein (G protein). The activation of the target protein either alters the concentration of one or more intracellular mediators (if the target protein is an enzyme) or alters the ion permeability of the plasma membrane (if the target protein is an ion channel). The intracellular mediators act in turn to alter the behavior of yet other proteins in the cell. All of the G-protein-linked receptors belong to a large superfamily of homologous, seven-pass transmembrane proteins.

G protein linked receptors are the largest family of cell surface receptors. More than 100 members have already been defined in mammals. G protein linked receptors mediate the cellular responses to an enormous diversity of signalling molecules, including hormones, neurotransmitters, and local mediators, which are as varied in structure as they are in function: the list includes proteins and small peptides, as well as amino acid and fatty acid derivatives. The same ligand can activate many different family members. At least 9 distinct G protein linked receptors are activated by adrenaline, for example, another 5 or more by acetylcholine, and at least 15 by serotonin. G protein linked receptors include, for example, the alpha-adrenergic receptors, the beta-adrenergic receptors, dopaminergic receptors, serotonergic receptors, muscarinic cholinergic receptors, peptidergic receptors, and the thyrotropin releasing hormone receptor.

Most G protein linked receptors activate a chain of events that alters the concentration of one or more small intracellular signalling molecules. These small molecules, often referred to as intracellular mediators (also called intracellular messengers or second messengers), in turn pass the signal on by altering the behavior of selected cellular proteins. Two of the most widely used intracellular mediators are cyclic AMP (cAMP) and Ca²⁺: changes in the concentrations are stimulated by distinct pathways in most animal cells, and most G-protein-linked receptors regulate one or the other of them.

The third type of cell surface receptor proteins are the enzyme linked receptors which, when activated, either function directly as enzymes or are associated with enzymes. Most are single-pass transmembrane proteins, with their ligand-binding site outside the cell and their catalytic site inside. Compared with the other two classes, enzyme linked receptors are heterogeneous, although the great majority are protein kinases, or are associated with protein kinases, that phosphorylate specific sets of proteins in the target cell.

There are five known classes of enzyme-linked receptors: (1) transmembrane guanylyl cyclases, which generate cyclic GMP directly; (2) receptor tyrosine phosphatases, which remove phosphate from phosphotyrosine side chains of specific proteins; (3) transmembrane receptor serine/threonine kinases, which add a phosphate group to serine and threonine side chains on target proteins; (4) receptor tyrosine kinases; and (5) tyrosine-kinase-associated receptors.

The last two types of receptors are by far the most numerous, and they are thought to work in a similar way: ligand binding usually induces the receptors to dimerize, which activates the kinase activity of either the receptor or its associated nonreceptor tyrosine kinase. When activated, receptor tyrosine kinases usually cross-phosphorylate themselves on multiple tyrosine residues, which then serve as docking sites for a small set of intracellular signaling proteins. In this way, a multiprotein signalling complex is activated from which the signal spreads to the cell interior.

In addition to these numerous types of cell surface signalling receptors, cell signalling is also accomplished via intracellular cell signalling receptors. As indicated above, these receptors are inside the target cell and the signalling ligand has to enter the cell to activate them. Steroid hormones, thyroid hormones, retinoids, and vitamin D are examples of small hydrophobic molecules that differ greatly from one another in both chemical structure and function. Nonetheless, they all act by a similar mechanism. They diffuse directly across the plasma membrane of target cells and bind to intracellular cell signalling receptors. Ligand binding activates the receptors, which then directly regulate the transcription of specific genes. These receptors are structurally related and constitute the intracellular receptor superfamily (or steroid-hormone receptor superfamily).

Steroid hormone receptors include progesterone receptors, estrogen receptors, androgen receptors, glucocorticoid receptors, and mineralocorticoid receptors. Thyroid hormone receptors include the thyroid stimulating hormone receptor. Retinoid receptors include the receptor for retinoic acid.

According to the subject invention, a cell is transformed with DNA encoding one or more of these cell signalling receptors which is not endogenous to the cell (which may be a cell surface signalling receptor or an intracellular cell signalling receptor). The non-endogenous receptor is capable of activating a signal transduction pathway that is endogenous to the cell. As used herein, a “non-endogenous” receptor is a receptor not normally present in/on (used interchangeably throughout this application) the particular cell. A receptor, therefore, may be non-endogenous to the transformed cell even though the receptor is present in/on other cells in the selected organism (i.e. in a human being). For example, a receptor present only in heart muscle tissue is a “non-endogenous” receptor in a liver cell. In contrast, an “endogenous” pathway as used herein refers to a signal transduction pathway that is normally present in the transformed cell. Similarly, “ectopic” expression as used herein refers to expression of a receptor in/on a particular cell in/on which the receptor is not normally present. “Eutopic” expression, in contrast, is expression of a receptor in/on a particular cell in/on which the receptor is normally present.

The DNA encoding the non-endogenous receptor is capable of activating an endogenous signal transduction pathway in the transformed cell. Signal transduction pathways are numerous, and include, for example, adenylate cyclase pathways, guanylate cyclase pathways, phosphoinositol turnover pathways, tyrosine kinase pathways, ion channel pathways, and calcium ion pathways. The initial signal produced by the binding of an extracellular signalling molecule to a cell signalling receptor is transduced intracellularly via a signal transduction pathway that may be multiple and interactive.

Signal transduction pathways involve cAMP, cGMP, arachidonic acid, inositol 1,4,5-tris-phosphate (IP₃), Ca²⁺, and other ions as second messengers and are produced by enzymes (such as adenylate and guanylate cyclases and phospholipases A₂ and C) and ion channels. In many cases, an extracellular molecule-receptor complex does not interface directly with these effectors but acts via an intermediate modulating signal transducer, often a G protein.

cAMP is the prototypical second messenger. Intracellular levels of cAMP are determined, in large part, by ligand-receptor interactions. This physiological event involves the interaction of three cellular components located near the plasma membrane: the ligand receptor, a signal transducer (G protein), and the effector enzyme (adenylate cyclase).

Cyclic AMP is a second messenger for many hormones, including epinephrine, glucagon, norepinephrine, parathyroid and luteinizing hormones, and thyroid-stimulating and melanocyte-stimulating hormones. Cyclic AMP affects a wide range of cellular processes: increases lipolysis, glycogen degradation, gluconeogenesis, ketogenesis, ion permeability of epithelia, renin production by kidney, contraction of cardiac muscle, HCl secretion by the gastric mucosa, amylase release by the parotid gland, dispersion of melanin granules, and insulin release by the pancreas; decreases aggregation of platelets, and growth of tumor cells in tissue culture.

The activation of adenyl cyclase leads to an increased amount of cyclic AMP inside the cell. Cyclic AMP then activates a protein kinase, which phosphorylates one or more proteins. For example, the phosphorylation of glycogen synthetase and phosphorylase kinase in muscle and liver results in decreased synthesis and enhanced degradation of glycogen. Table 1 classifies various hormone receptors and effectors according to the pathway utilized.

Several receptors contain an intrinsic hormone-activated tyrosine kinase activity.

Guanylate cyclase catalyzes the formation of cGMP from GTP, in analogy with adenylate cyclase. Guanylate cyclase, however, exists in cells in both soluble and membrane-associated forms. The membrane associated form is regulated by hormones and other ligands. Like the tyrosine kinases and unlike adenylate cyclase, guanylate cyclase may directly serve receptor and effector functions.

A number of hormones and ligands mediate their cellular actions via calcium ions and DAG as second messengers. The second messengers, in turn, modulate the activity of protein kinases regulated by calcium binding-regulatory protein (e.g. calmodulin), and DAG activates protein kinase C. These enzymes phosphorylate specific intracellular proteins, which results in further hormone action. Example of hormones using this signaling system in specific tissues include alpha-1-adrenergic and muscarinic cholinergic agents, vasopressin, histamine, cholecystokinin, LHRH, thyrotropin-releasing hormone, angiotensin II, and oxytocin.

In general, the various hormone ligands that stimulate phosphoinositide turnover interact with the receptors that activate G proteins, as described for adenylate cyclase. These activated G proteins, however, are coupled to stimulation of phospholipase C activity.

Depending upon the pathway utilized, many different cellular functions are controlled by these various pathways. Numerous hormone-induced cellular responses are mediated by cyclic AMP, including: in the thyroid gland, thyroid-stimulating hormone (TSH) induces thyroid hormone synthesis and secretion; in the adrenal cortex, adrenocorticotropic hormone (ACTH) induces cortisol secret ion; in the ovary, luteinizing hormone (LH) induces progesterone secretion; in muscle, adrenaline induces glycogen breakdown; in bone, parathormone induces bone resorption; in the heart, adrenaline induces an increase in heart rate and force of contraction; in the liver, glucagon induces glycogen breakdown; in the kidney, vasopressin induces water resorption; and in fat, adrenaline, ACTH, glucagon, and TSH induce triglyceride breakdown.

TABLE 1
Classification of Hormone Receptors and Effectors

Adenylate cyclase
Beta-adrenergic catecholamines
Luteinizing hormone and human chorionic gonadotropin
Follicle-stimulating hormone Corticotropin
Prostaglandins Parathyroid hormone
Alpha-adrenergic (inhibition) TSH
Somatostatin (inhibition)g Glucagon
Guanylate Cyclase
Atrial peptide (AP, also called atrial natriuretic factor)
Receptor Protein Tyrosine Kinases
Insulin
Insulin-like growth factor (somatomedin-C)
Epidermal growth factor
Colony-stimulating factor 1
Fibroblast growth factor
Platelet-derived growth factor
Phosphoinositol Turnover and Calcium Flux
Acetylcholine receptor (muscarinic)
Alpha-adrenergic catecholamines Angiotensin
Luteinizing hormone-releasing hormone Vasopressin
Thyrotropin-releasing hormone
Ion Channels
Acetylcholine receptor (nicotinic) Glycine
Gamma-aminobutyric acid Kainate
Sodium Calcium Potassium
Unknown Effector System
Growth hormone Prolactin
Erythropoietin Interleukins
Nerve growth factor T cell receptor

Numerous cellular responses are mediated by G-protein-linked receptors coupled to the inositolphospholipid signalling pathway, including: in the liver, the signalling molecule vasopressin induces glycogen breakdown; in the pancreas, the signalling molecule acetylcholine induces amylase secretion; in smooth muscle, the signalling molecule acetylcholine induces contraction; in mast cells, an antigen functions as a signalling molecule to induce histamine secretion; and in blood platelets, the signalling molecule thrombin induces aggregation.

These various cellular functions can thus be controlled by transforming a cell with DNA encoding a non-endogenous cell signalling receptor which is capable of activating one or more of these endogenous signal transduction pathways.

For a general discussion of cell signalling, see references (37-42).

In addition to selecting a non-endogenous receptor and an endogenous signal transduction pathway, a method for transforming cells must also be selected. Various methods are known in the art. One of the first methods was microinjection, in which DNA was injected directly into the nucleus of cells through fine glass needles. This was an efficient process on a per cell basis, that is, a large fraction of the injected cells actually got the DNA, but only a few hundred cells could be injected in a single experiment.

The earliest method for introducing DNA into cells en masse was to incubate the DNA with an inert carbohydrate polymer (dextran) to which a positively charged chemical group (DEAE, for diethylaminoethyl) had been coupled. The DNA sticks to the DEAE-dextran via its negatively charged phosphate groups. These large DNA-containing particles stick in turn to the surfaces of cells, which are thought to take them in by a process known as endocytosis. Some of the DNA evades destruction in the cytoplasm of the cell and escapes to the nucleus, where it can be transcribed into RNA like any other gene in the cell.

The DEAE-dextran method, while relatively simple, was very inefficient for many types of cells, so it was not a reliable method for the routine assay of the biological activity of a purified DNA preparation. The breakthrough that eventually made gene transfer a routine tool for workers studying mammalian cells was the discovery that cells efficiently took in DNA in the form of a precipitate with calcium phosphate. With this new method, the yield of virus from cells transfected with viral DNA was a hundred times greater than with the DEAE-dextran method.

Biological markers can be used to identify the cells carrying recombinant DNA molecules. In bacteria, these are commonly drug-resistance genes. Drug resistance is used to select bacteria that have taken up cloned DNA from the much larger population of bacteria that have not. In the early mammalian gene transfer experiments involving viral genes, the transfer of exogenous DNA into cells was detected because the DNA had a biological activity, it led to production of infectious virus or produced stable changes in the growth properties of the transfected cells. It was then discovered that the DNA tumor virus, herpes simplex virus (HSV), contained a gene encoding the enzyme thymidine kinase (the tk gene). The HSV tk gene can be used as a selectable genetic marker in mammalian cells in much the same way that drug-resistance genes worked in bacteria, to allow rare transfected cells to grow up out of a much larger population that did not take up any DNA. The cells are transferred to selective growth medium, which permits growth only of cells that took up a functional tk gene (and the transferred DNA of interest).Various dominant selectable markers are now known in the art, including:

aminoglycoside phosphotransferase (APH), using the drug G418 for selection which inhibits protein synthesis; the APH inactivates G418;

dihydrofolate reductase (DHFR):Mtx-resistant variant, using the drug methotrexate (Mtx) for selection which inhibits DHFR; the variant DHFR is resistant to Mtx;

hygromycin-B-phosphotransferase (HPH), using the drug hygromycin-B which inhibits protein synthesis; the HPH inactivates hygromycin B;

thymidine kinase (TK), using the drug aminopterin which inhibits de novo purine and thymidylate synthesis; the TK synthesizes thymidylate;

xanthine-guanine phosphoribosyltransferase (XGPRT), using the drug mycophenolic acid which inhibits de novo GMP synthesis; XGPRT synthesizes GMP from xanthine; and

adenosine deaminase (ADA), using the drug 9-β-D-xylofuranosyl adenine (Xyl-A) which damages DNA; the ADA inactivates Xyl-A.

Gene amplification can also be used to obtain very high levels of expression of transfected gene. When cell cultures are treated with Mtx, an inhibitor of a critical metabolic enzyme, DHFR, most cells die, but eventually some Mtx-resistant cells grow up. A gene to be expressed in cells is cotransfected with a cloned dhfr gene, and the transfected cells are subjected to selection with a low concentration of Mtx. Resistant cells that have taken up the dhfr gene (and, in most cases, the cotransfected gene) multiply. Increasing the concentration of Mtx in the growth medium in small steps generates populations of cells that have progressively amplified the dhfr gene, together with linked DNA. Although this process takes several months, the resulting cell cultures capable of growing in the highest Mtx concentrations will have stably amplified the DNA encompassing the dhfr gene a hundredfold or more, leading to significant elevation of the expression of the cotransfected gene.

Although calcium phosphate coprecipitation is the most widely used method for introducing DNA into mammalian cells, in some cells it doesn't work. Cells such as lymphocytes, which grow in suspension, are especially resistant to transfection by calcium phosphate precipitates.

In another method, electroporation, cells are placed in a solution containing DNA and subjected to a brief electrical pulse that causes holes to open transiently in their membranes. DNA enters through the holes directly into the cytoplasm, bypassing the endocytotic vesicles through which they pass in the DEAE-dextran and calcium phosphate procedures (passage through these vesicles may sometimes destroy or damage DNA). DNA can also be incorporated into artificial lipid vesicles, liposomes, which fuse with the cell membrane, delivering their contents directly into the cytoplasm. Microinjection, the surest way to get DNA into cells, can now be performed with a computer-assisted apparatus that increases by 10-fold or more the number of cells that can be injected in one experiment. And in an even more direct approach, used primarily with plant cells and tissues, DNA is absorbed to the surface of tungsten microprojectiles and fired into cells with a device resembling a shotgun.

Several of these methods, microinjection, electroporation, and liposome fusion, have been adapted to introduce proteins into cells. For review, see references (47-50).

Although naked DNA introduced by transfection can be transiently expressed in up to half the cells in a culture, more frequently the fraction of transiently transfected cells is much lower. In fact, some cells are almost completely refractory to transfection by the artificial methods described above. Many applications of recombinant DNA technology require introducing foreign genes (i.e. non-endogenous receptors) into recalcitrant cell types. Potential gene therapy strategies, for example, require efficient means for transferring genes into normal human cells.

To solve this problem, researchers have turned to viruses. Viral growth depends on the ability to get the viral genome into cells, and viruses have devised clever and efficient methods for doing it. The earliest viral vectors, based on the monkey tumor virus SV40, simply substituted some of the viral genes with the foreign gene. These recombinant molecules, prepared as bacterial plasmids, were transfected into monkey cells together with a second plasmid that supplied the missing viral genes. Once inside the cells, viral gene products produced from the two plasmids cooperate to replicate both plasmids and package each into virus particles. The virus stock that emerges from the cell is a mixture of two viruses, each of which is by itself defective (that is, it cannot replicate on its own because it is missing necessary viral genes). Nevertheless, this virus stock can then be used to infect new cells, efficiently introducing and expressing the foreign gene in the recipient cells.

A hybrid method that uses transfection to get DNA into cells and a viral protein to replicate it once inside is now commonly used for high-level production of protein from a cloned gene. This procedure uses a cell line, COS cells, carrying a stably integrated portion of the SV40 genome. These cells produce the viral T antigen protein, which triggers replication of viral DNA by binding to a DNA sequence termed the origin of replication. The foreign gene to be expressed is cloned into a plasmid that carries the SV40 origin of replication. After transfection into COS cells, the plasmid is replicated to a very high number of copies, increasing the expression level of the foreign gene.

Use of SV-40-based viral vectors is limited for a number of reasons: they infect only monkey cells, the size of foreign gene that can be inserted is small, and the genomes are often rearranged or deleted. Other viral vectors are more commonly used now, either because they can infect a wider range of cells or because they accept a wider range of foreign genes. Vaccinia virus is a large DNA-containing virus that replicates entirely in the cytoplasm. Early vaccinia vectors incorporated the foreign gene directly into a nonessential region of the viral genome. Recombinant viruses are viable and upon infection transcribe the foreign gene from a nearby viral promoter. Because the viral genome is large (185,000 bp), foreign genes cannot be inserted into vaccinia by standard recombinant DNA methods; instead, it must be done by recombination inside cells, a cumbersome and lengthy procedure. A more versatile vaccinia expression system uses a ready-made recombinant virus that expresses a bacteriophage RNA polymerase. The gene to be expressed is simply cloned into a plasmid carrying a bacteriophage promoter. The plasmid is transfected into cells that have been previously infected with the vaccinia virus that expresses the RNA polymerase. The gene on the plasmid is efficiently transcribed by the bacteriophage polymerase, accounting for up to 30 percent of the RNA in the cell. An additional feature of vaccinia virus infection is that the virus shuts down host cell protein synthesis so that viral mRNA (and mRNA from the plasmid) are preferentially translated into protein.

Another virus widely used for protein production is an insect virus, baculovirus. Baculovirus attracted the attention of researchers because during infection, it produces one of its structural proteins (the coat protein) to spectacular levels. If a foreign gene were to be substituted for this viral gene, it too ought to be produced at high levels. Baculovirus, like vaccinia, is very large, and therefore foreign genes must be placed in the viral genome by recombination. To express a foreign gene in baculovirus, the gene of interest is cloned in place of the viral coat protein gene in a plasmid carrying a small portion of the viral genome. The recombinant plasmid is cotransfected into insect cells with wildtype baculovirus DNA. At a low frequency, the plasmid and viral DNAs recombine through homologous sequences, resulting in the insertion of the foreign gene into the viral genome. Virus plaques develop, and the plaques containing recombinant virus look different because they lack the coat protein. The plaques with recombinant virus are picked and expanded. This virus stock is then used to infect a fresh culture of insect cells, resulting in high expression of the foreign protein. For a review of baculovirus vectors, see reference (51).

All of the viruses discussed above are lytic viruses, in that they enter cells, take over, replicate massively, and get out, killing the cell in the process. So these vectors cannot be used to introduce a gene into cells in a stable fashion. This task is most ably performed by retroviruses. Retroviruses are RNA viruses with a life cycle quite different from that of the lytic viruses. When they infect cells, their RNA genomes are converted to a DNA form (by the viral enzyme reverse transcriptase). The viral DNA is efficiently integrated into the host genome, where it permanently resides, replicating along with host DNA at each cell division. This integrated provirus steadily produces viral RNA from a strong promoter located at the end of the genome (in a sequence called the long terminal repeat or LTR).This viral RNA serves both as mRNA for the production of viral proteins and as genomic RNA for new viruses. Viruses are assembled in the cytoplasm and bud from the cell membrane, usually with little effect on the cell's health. Thus, the retrovirus genome becomes a permanent part of the host cell genome, and any foreign gene placed in a retrovirus ought to be expressed in the cells indefinitely.

Retroviruses are therefore attractive vectors because they can permanently express a foreign gene in cells. Moreover, they can infect virtually every type of mammalian cell, making them exceptionally versatile. Because of their versatility, retroviruses are also the vector of choice for gene therapy. In the design and use of retroviral vectors, the vectors usually contain a selectable marker as well as the foreign gene to be expressed. Most of the viral structural genes are gone, so these vectors cannot replicate as viruses on their own. To prepare virus stocks, cloned proviral DNA is transfected into a packaging cell. These cells usually contain an integrated provirus with all its genes intact, but lacking the sequence recognized by the packaging apparatus. Thus, the packaging provirus produces all the proteins required for packaging of viral RNA into infectious virus particles but it cannot package its own RNA. Instead, RNA transcribed from the transfected vector is packaged into infectious virus particles and released from the cell. The resulting virus stock is termed helper-free because it lacks wild-type replication-competent virus. This virus stock can be used to infect a target cell culture. The recombinant genome is efficiently introduced, reverse transcribed into DNA (by reverse transcriptase deposited in the virus by the packaging cells), and integrated into the genome. Thus, the cells now express the new virally introduced gene, but they never produce any virus, because the recombinant virus genome lacks the necessary viral genes. For a review of retrovirus vectors, see references (52,53).

Another viral vector is adenovirus, reviewed by Berkner, K. L. (54). Still another viral vector is herpesvirus.

As indicated, some of these methods of transforming a cell require the use of an intermediate plasmid vector. U.S. Pat. No. 4,237,224 (Cohen et al.) describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture. The DNA sequences are cloned into the plasmid vector using standard cloning procedures in the art, as described by Maniatis et al., Molecular Cloning: A Laboratory Manual (Cold Springs Laboratory, Cold Springs Harbor, N.Y. 1982).

A transformed cell containing a foreign gene of interest (i.e., a non-endogenous receptor) can then be utilized for gene therapy purposes. Note that the cell may be transformed in vitro and reinserted into a multicellular organism, or the cell may be transformed in vivo. In the same way that a retrovirus acts as a vector to carry a gene into a cell, so the cell can be regarded as a vector for carrying the gene into a patient's body.

Suitable cells for transformation and use in gene therapy should be readily obtainable, grow well in culture, and be able to withstand the various manipulations involved in, for example, retrovirus or adenovirus infection. For in vitro transformation, vector cells should be easy to return to the patient after such transformation and should continue to live for many months, preferably for the life of the patient. See Friedmann (55), Verma (56), Anderson (62) and Mulligan (63) for discussions of gene therapy.

The cells of the bone marrow have many of these desirable features. The bone marrow contains stem cells that give rise to all cells of the hematopoietic series. Infection of these stem cells results in a continuous supply of cells containing the therapeutic gene. Furthermore, the techniques for reconstituting the bone marrow of patients and experimental animals are well worked out. Convincing evidence that genes introduced in hematopoietic stem cells are expressed and that they can produce a therapeutic effect has come from experiments with a mutant dihydrofolate reductase gene (DHFR). See Corey et al. (57).

Another cell type that has been studied intensively as a vehicle for gene transfer is the skin fibroblast. Fibroblasts are easily obtainable, grow well in culture, and have been the subjects of many experiments, and skin fibroblasts can be efficiently infected with viral vectors, such as retroviral vectors. See Palmer et al. (58).

Another target tissue for gene therapy is the liver. A large number of inherited metabolic disorders affect the liver, and liver transplantation has been tried in an effort to treat these and other conditions such as hypercholesterolemia and hemophilia. Techniques have been developed for isolating and culturing hepatocytes, so the appropriate target cells are available for viral-mediated gene transfer. See Anderson et al. (59).

One way to avoid the complications of developing cell-based systems for delivering genes to patients is to deliver the viral vectors directly to the target cells. This technique has been shown to be an efficient way to infect the endothelial cells of blood vessel walls using retroviral vectors. See Nabel et al. (60) and Nabel et al. (61).

As indicated above, suitable cells to be transformed according to the subject invention include, for example, a liver cell (known as a hepatocyte) in which one desires to control glycogenolysis. This can be accomplished by transforming the liver cell with DNA encoding a cell signalling receptor not endogenous to the cell (i.e. not normally present on the liver cell), where the receptor is capable of activating a signal transduction pathway endogenous (i.e. normally present in the cell) to the liver cell. The non-endogenous receptor is chosen such that the compatible signal transduction pathway regulates glycogenolysis in the cell. In the example of a liver cell, the cAMP pathway and phosphoinositide pathways within the liver cell regulate glycogenolysis, and DNA encoding a thyrotropin releasing hormone receptor (a receptor not endogenous to the liver cell) can be transformed into the liver cell to activate these two endogenous pathways, thereby providing for controllable activation of the pathways (by introducing an extracellular molecule capable of binding to the non-endogenous receptor and thereby stimulating the endogenous pathway).

A liver cell is one example of a suitable in vivo cell according to the subject invention. Other cells are equally transformable, such as cells of the pulmonary airways. These cells can be transformed with a thyroid-stimulating hormone receptor which can utilize the pulmonary airways' endogenous cyclic AMP signal transduction pathway. Extracellular molecules capable of activating the thyroid-stimulating hormone receptor would therefore control the same cellular function as do adrenergic agonists and bronchodilating drugs (which utilize the cAMP pathway to normally control cellular function).

The numerous other suitable cells would be apparent to those skilled in the art.

The particular examples which follow utilize adenovirus-based vectors to transform liver cells. Adenovirus based vectors have been used successfully to express a number of mammalian and viral proteins but they have not been used to ectopically express cell signalling receptors in vivo. The following examples show that functions of a specific cell type can be regulated in an intact animal by an agonist that does not usually affect functions in this cell type (because the cell does not endogenously express the receptor for this agonist) when the receptor for this agonist is expressed by adenovirus-mediated gene transfer. This is so because the expressed non-endogenous receptor couples to an endogenous signal transduction pathway and thereby regulates cell function.

As discussed below, receptors for the neurohormone thyrotropin-releasing hormone (TRH) were expressed by adenovirus-mediated gene transfer in the livers of intact rats. When administered to these rats but not to uninfected rats or to rats infected with a null virus, TRH caused an elevation of blood sugar. This rise in blood sugar is similar to that observed when vasopressin is given to rats because hepatocytes endogenously express receptors for vasopressin that signal through the same signal transduction system as the TRH receptor. In this manner, any receptor ectopically expressed in any cell type may be made to control functions of that cell.

This invention has important therapeutic applications. These applications include temporary relief of life-threatening illnesses, with receptor expression in diseased tissues and in tissues unaffected by the disease process. An example of an application in a diseased tissue would be the expression of the receptor for thyroid-stimulating hormone in pulmonary airways to mediate, via an elevation of cyclic AMP, which is the same pathway, used by adrenergic agonists and bronchodilating drugs, a bronchodilatory response in patients with chronic obstructive pulmonary disease or asthma suffering a marked exacerbation. A therapeutic advantage of using more than one receptor to mediate the desired response is that the efficacy of most drugs decreases upon repeated administrations, a process termed tachyphylaxis, which is usually caused by receptor desensitization. By activating different receptors at different times, one would anticipate a better therapeutic response. For example, in the patient suffering an episode of marked bronchoconstriction as described above, one would predict a better response to thyroid-stimulating hormone than to adrenergic agonists if adrenergic agonists had been used chronically.

Another application is for the treatment of diseases for which regulation of a physiologic function in a healthy organ would counter-balance the effects of a disease process. An example of this is presented in the Examples which follow, in which blood glucose was elevated by TRH after ectopic expression of TRH receptors in liver. This approach can be used in a patient with severe hypoglycemia who harbors an inoperable insulin-secreting tumor (insulinoma) to maintain normoglycemia over an extended period of time. In such a patient it might be better to ectopically express a non-endogenous receptor that signals via the cAMP transduction system, such as the receptor for thyroid-stimulating hormone, as glycogenolysis in human liver is more effectively regulated by this pathway than by the phosphoinositide pathway to which TRH receptors couple. It is important to note that it would be better to use a non-peptide agonist drug rather than the natural peptide agonist so that receptor activation could be accomplished by an oral medication.

Maintenance of blood glucose by glycogenolysis in the liver is normally initiated by extracellular regulatory molecules such as glucagon and vasopressin triggering specific receptors on hepatocytes to activate the cAMP or phosphoinositide signal transduction pathways. The subject invention demonstrates that the normal ligand-receptor regulators of glycogenolysis can be bypassed using an adenovirus vector to ectopically express the non-endogenous mouse pituitary thyrotropin releasing hormone receptor (TRH-R) cDNA in rat liver in vivo. The ectopically expressed TRH-R links to the phosphoinositide pathway, providing a means to activate glycogenolysis with TRH, an extracellular ligand not normally associated with liver physiology. When TRH is administered to these animals, the phosphoinositide path is activated resulting in a sustained rise in blood glucose.

Since activation of a specific signal transduction pathway in a differentiated cell will trigger a cell-specific response independent of the ligands and receptors by which the signal transduction pathway is normally activated (8,9), the invention provides an approach to the therapy of such disorders which is to bypass the normal extracellular regulatory molecule and its specific endogenous receptor by using an alternative ligand and non-endogenous receptor to trigger the relevant endogenous signal transduction pathway. Such a strategy can be used to correct abnormalities of extracellular regulatory molecules or their specific endogenous receptors, as well as to modulate specific physiologic functions in disease states by re-establishing control of specific signal transduction pathways.

The subject invention provides a new strategy to specifically control the differentiated function of cells in vivo by providing the means to trigger a specific signal transduction pathway by using a gene transfer vector to ectopically express a naturally occurring receptor in cells of an organ that do not normally express that receptor (i.e. a non-endogenous receptor). The expressed receptor links to its natural signal transduction pathway, enabling specific responses to be triggered in the target cells by a ligand relevant for that receptor. For in vitro data, see references (10-12).

In the examples which follow, the consequences of systemic administration of thyrotropin-releasing hormone (TRH) after adenovirus-mediated transfer and ectopic expression of the thyrotropin-releasing hormone receptor (TRH-R) cDNA in hepatocytes in vivo is shown. The TRH-R is a seven transmembrane spanning receptor normally expressed in the anterior pituitary gland, and not in hepatocytes (13). When TRH-Rs in pituitary cells are stimulated by TRH, the TRH-R triggers, via a guanine nucleotide binding protein, the phosphoinositide-calcium-protein kinase C signal transduction pathway and the pituitary releases thyroid stimulating hormone (TSH) (2,13,14). In contrast, triggering of the phosphoinositide pathway in hepatocytes (for example, with vasopressin) results not in TSH release, but in glycogen breakdown and the release of glucose into the circulation (5).

The subject invention provides for the transfer and expression of the TRH-R cDNA in hepatocytes in vivo with linkage of the expressed receptor to the hepatocyte phosphoinositide pathway. Therefore, systemic administration of TRH to animals expressing TRH receptors in the liver triggers the phosphoinositide pathway in hepatocytes, resulting in a rise in blood glucose.

### EXAMPLE 1

### Construction of AdCMV.m TRH-R

The parent plasmid, pAdCMV.mTRH-R, was constructed by inserting a 1.2 kb EcoRI-NotI fragment containing the protein-coding region of the mouse TRH-R cDNA, nucleotides 233-1462 of plasmid pBSmTRHR (43), into plasmid pGEM2-L3-114 at the EcoRI-Bam-HI site. After digesting with EcoRI and using the Klenow fragment of DNA polymerase I to make blunt DNA ends, HindIII linkers were ligated and a 1.4 kb HindIII fragment containing mouse TRH-R cDNA and the adenovirus E2 poly(A) signal sequence was isolated and inserted into the HindIII site of the pAdCMV-HS-Vector which contains the left end replication and packaging elements of adenovirus, the cytomegalovirus-1 promoter and splicing elements from plasmid pML-IS Cat (44). Following verification of the plasmid by restriction site mapping and transient transfection of pAdCMV.mTRH-R into COS-1 cells to demonstrate TRH-R expression, the virus AdCMV.mTRH-R was constructed by overlap recombination as described by Tantravahi et al. (45). All transfections were carried out in human embryonic kidney cells transformed with the El region of adenovirus type 5 according to the procedure of Graham et al. (46). Following plaque purification, virus was grown in 293 cells in suspension cultures as described by Tantravahi et al. (45). The entire sequence coding for the adenovirus E1a gene was removed as well as the 5′1.8 kb of the E1b gene. Co-transfection of pAdCMV.mTRH-R with the large fragment of adenovirus (3.8-100 map units) into 293 cells resulted in production of recombinant virus AdCMVmTRHR.

The construction of the recombinant plasmid designated pAdCMV.mTRH-R and the adenovirus vector designated AdCMV.mTHR-R is described fully by Gershengorn et al. (10) and Falck-Pedersen et al. (30).

### EXAMPLE 2

The ability of AdCMV.mTRH-R to transfer and express the mouse TRH-R cDNA in rat hepatocytes was first demonstrated in vitro. The replication-deficient recombinant adenovirus vectors are all E1a⁻, partial E1b⁻, partial E3⁻ based on adenovirus type 5, in which an expression cassette containing a promoter, driving the expression of a recombinant gene, is inserted at the site of the El deletion. AdCMV.mTRH-R contains an expression cassette with the cytomegalovirus early intermediate promoter/enhancer (CMV) followed by the mouse thyrotropin releasing hormone receptor (TRH-R) cDNA (30). AdCMV.βgal carries the CMV promoter and the E.coli LacZ gene [encoding β-galactosidase (βgal)] (31). The adenovirus vectors were prepared, purified, and titered as previously described (32,33). Primary hepatocyte cultures established from 250-300 g male Sprague-Dawley rats (19), were exposed to AdCMV.mTRH-R at different multiplicity of infection (moi, 1, 10, 50, 100); uninfected cells and cells infected with AdCMV.βgal (moi 50) were used as controls.

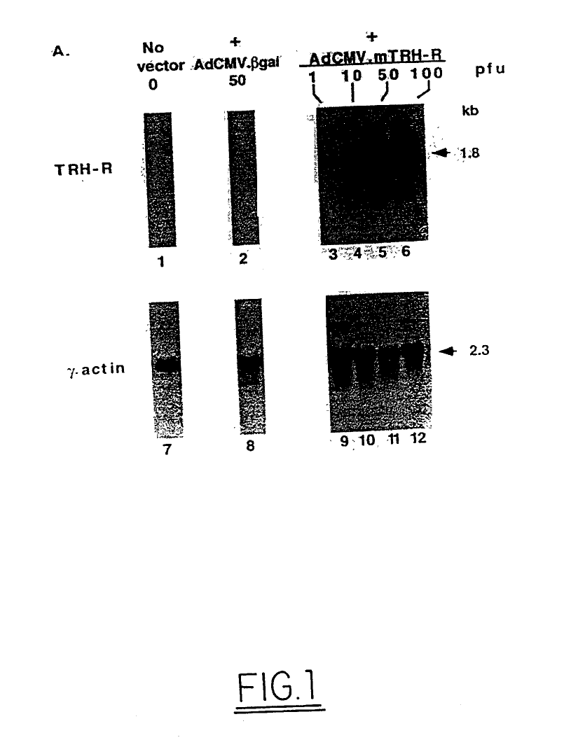

As shown in the Northern analysis of FIG. 1, RNA (10 μg/lane) from primary hepatocytes was evaluated 24 hr after incubation with the vectors using a mouse TRH-R cDNA probe (lanes 1-6), or as a positive control, human γ-actin cDNA probe (lanes 7-12). The sizes of the transcripts are indicated. Incubation of the hepatocytes with AdCMV.mTRH-R resulted in a dose-dependent expression of TRH-R mRNA transcripts (see FIG. 1, lanes 3-6). In contrast, hepatocytes that were uninfected or infected with the control vector AdCMV.βgal demonstrated no TRH-R mRNA transcripts (see FIG. 1, lanes 1 and 2), although control γ-actin mRNA transcripts were similar in all samples (see FIG. 1, lanes 7-12).

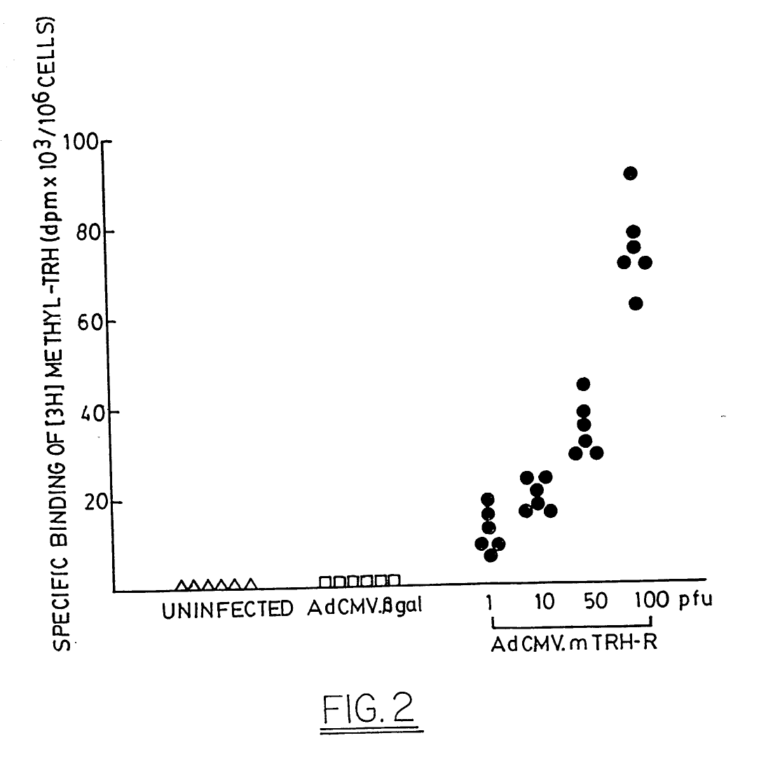

FIG. 2 illustrates the binding of methylthyrotropin releasing hormone (methyl-TRH) to hepatocytes following adenovirus-mediated in vitro transfer of the TRH-R cDNA. Preliminary studies demonstrated that methyl-TRH binding had a Kd of 3.18±0.39 nM; based on this, 1 nM methyl-TRH was used for the binding studies. Following incubation for 24 hr with the vectors AdCMV.βgal or AdCMV.mTRH-R, the hepatocytes (10⁶/well) were incubated for 60 min with [³H]methyl-TRH (82.5 Ci/mmol, New England Nuclear-Dupont, Boston, Mass.) or [³H]methyl-TRH plus excess unlabeled methyl-TRH (Sigma, St. Louis, Mo.). Free ligand was removed by aspirating the medium and washing the cells 5 times with 2 ml (4° C.) Hanks balanced salt solution. Cell associated radioactivity was measured by dissolving the cells with 1 ml of 0.4 N NaOH and counting. Specific binding of the [³H]methyl-TRH was calculated as: [(dpm of [³H]methyl-TRH)-(dpM [³ H]methyl-TRH in the presence of unlabeled methyl-TRH)] (34). For each dose of the vector, triplicate measurements were made in hepatocytes from two different animals. The number of TRH-R receptor sites was calculated assuming a one-to-one stoichiometry of ligand to receptor and a homogeneous distribution of TRH-Rs. Addition of labeled methyl-TRH [an analog with higher affinity than TRH (15)], demonstrated specific binding of methyl-TRH, with the amount of binding dependent on the dose of AdCMV.mTRH-R used to infect the hepatocytes (see FIG. 2). In contrast, analysis of hepatocytes that were uninfected or infected with the control vector AdCMV.βgal showed no specific binding of methyl-TRH (see FIG. 2). Quantification of the number of binding sites of methyl-TRH demonstrated 5.7×10⁵ sites/cell with a Kd of 3.18±0.39 nM (AdCMV.mTRH-R 50 moi). Following addition of methyl-TRH to the hepatocytes infected with AdCMV.mTRH-R, the ligand-receptor complex was internalized (not shown).

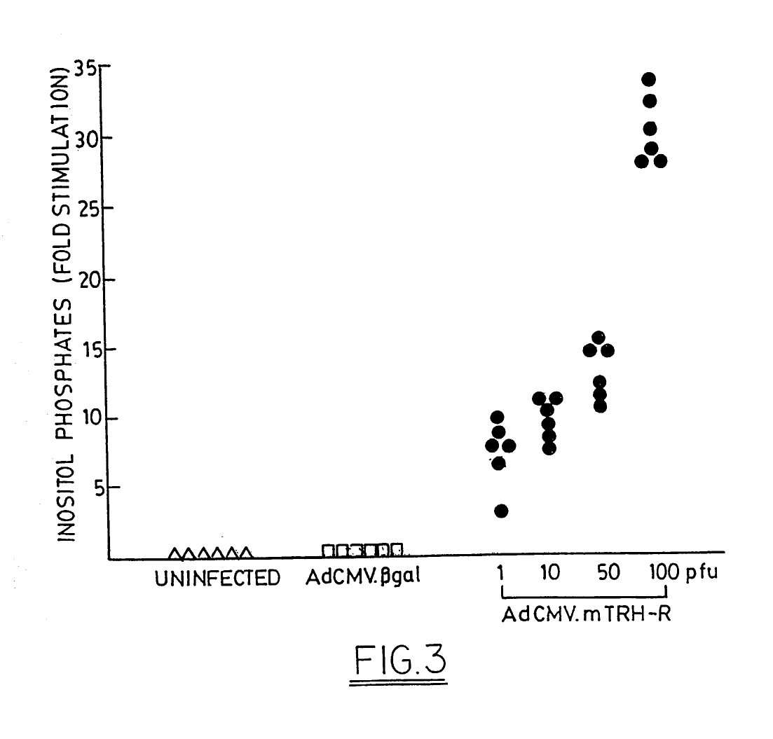

FIG. 3 shows the TRH stimulation of inositol phosphate formation in hepatocytes following adenovirus-mediated in vitro transfer of the TRH-R cDNA. Primary hepatocytes were exposed to the vectors AdCMV.βgal or AdCMV.mTRH-R. After 24 hr, the cells were labeled for 24 hr with myo-[³H]inositol (myo-[2-³H]inositol; 20 Ci/mmol, Amersham Corporation, Arlington Heights, Ill.), incubated (5 min, 37° C.) with 10 mM LiCl, stimulated (60 min, 37° C.) with methyl-TRH. The cells were then lysed, the inositol phosphates were separated by anion-exchange chromatography and the radioactivity counted. The fold stimulation of inositol phosphates formation was calculated as: (dpm in inositol phosphates ×100/dpm in lipids of stimulated cells) (dpm in inositol phosphates ×100/dpm in lipids of unstimulated cells) (35). For each dose of vector, triplicate measurements were made in hepatocytes from two different animals.

Direct evidence of specific activation of the phosphoinositide pathway by TRH in AdCMV.mTRH-R infected hepatocytes was the demonstration of increased amounts of inositol phosphates in the hepatocytes infected with AdCMV.mTRH-R and stimulated with methyl-TRH (see FIG. 3). Importantly, the increase in inositol phosphates was dependent on the dose of AdCMV.mTRH-R used to infect the cells, whereas hepatocytes that were uninfected, as well as hepatocytes infected with the control vector AdCMV.62 gal, showed no increase in inositol phosphates when incubated with methyl-TRH.

### EXAMPLE 3

As observed in vitro, the AdCMV.mTRH-R vector effectively transferred the TRH-R cDNA to the rat liver in vivo, with the consequent ectopic expression of functional TRH-Rs in the hepatocytes. To accomplish this, the AdCMV.mTRH-R was administered intravenously to the animals, a route of administration of replication deficient adenovirus vectors that results in >90% detected expression of the exogenous gene in hepatocytes (16).

Sprague-Dawley rats (250-300 g, 3 months old, fed ad libitum) were anaesthetized with ketamine-HCl (60 mg/kg) and xylazine (5 mg/kg). The Ad vectors [AdCMV.mTRH-R, AdCMV.βgal, AdCMV.Null (the “Null” vector is similar to the other vectors except that the expression cassette contains the CMV promoter but no recombinant gene) (31)] were administered via the right external jugular vein (5×10⁹ pfu, 100 μl 0.9% NaCl). The livers were recovered 5 days later.

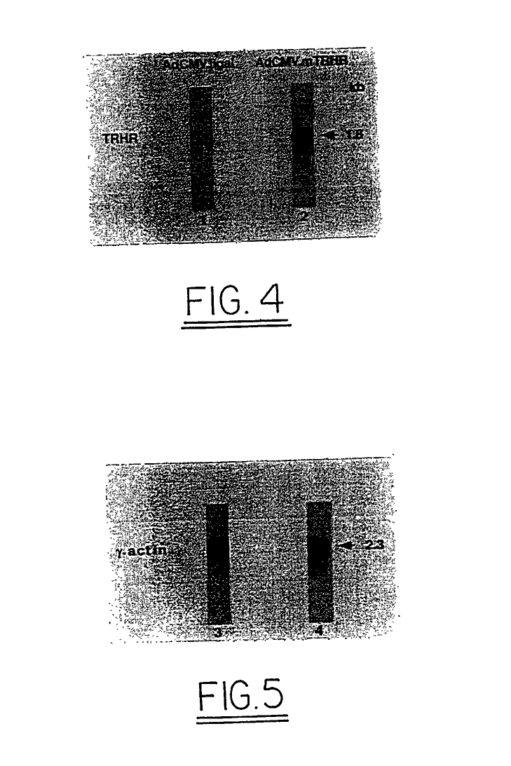

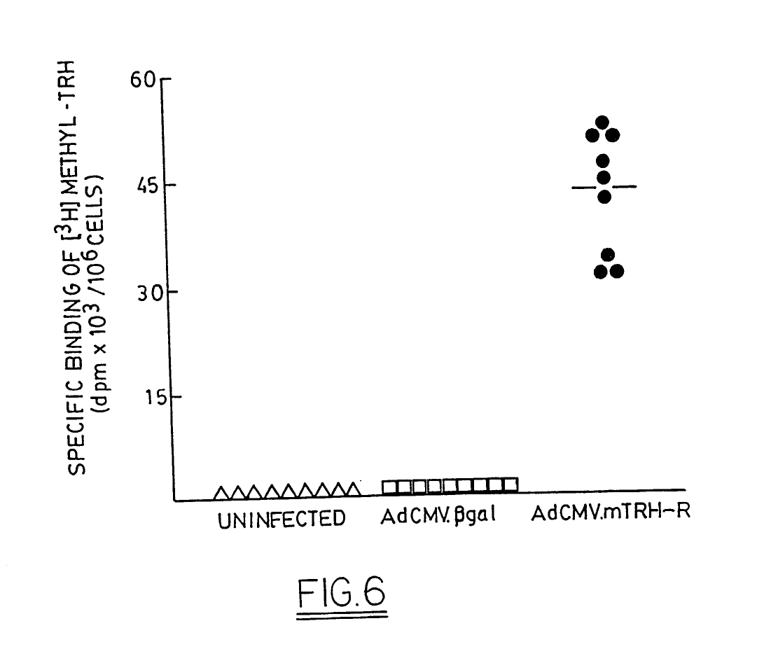

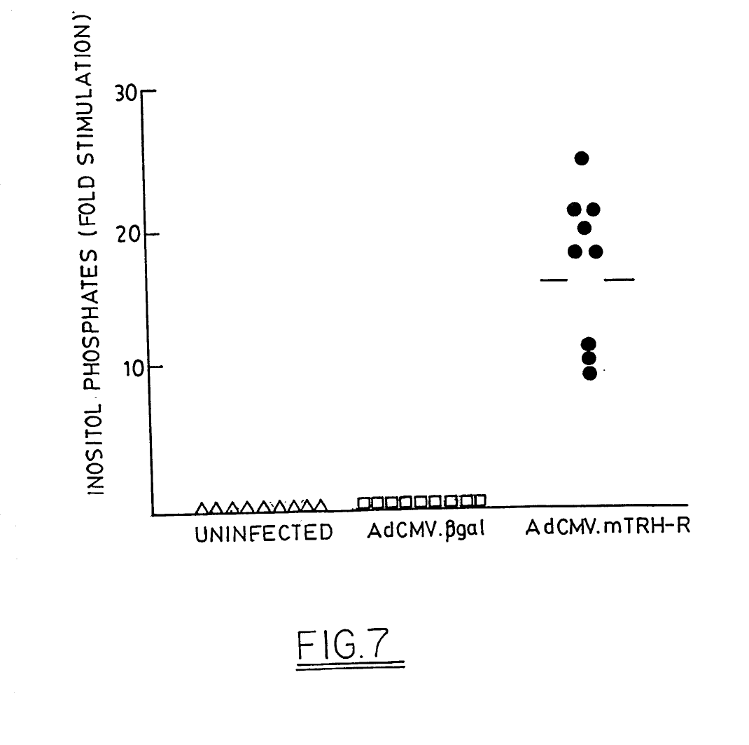

Cultures of primary hepatocytes from animals infected in vivo 5 days previously with AdCMV.mTRH-R demonstrated TRH-R mRNA transcripts, the ability to specifically bind TRH and the activation of the phosphoinositide pathway (FIGS. 4-7). FIGS. 4 and 5 are Northern analyses. Liver RNA (10 μg/lane) was evaluated with a mouse TRH-R cDNA probe (FIG. 4, lanes 1 and 2) or, as a positive control, W-actin cDNA (FIG. 5, lanes 3 and 4). The sizes of the transcripts are indicated. Lanes 1 and 3 (FIG. 4 and FIG. 5, respectively) are animals receiving AdCMV. βgal. Lanes 2 and 4 (FIG. 4 and FIG. 5, respectively) are animals receiving AdCMV.mTRH-R. Northern analysis of hepatocytes obtained from animals receiving the control vector AdCMV.βgal demonstrated no TRH-R mRNA specific transcripts (FIG. 4, lane 1 and FIG. 5, lane 3). In contrast, hepatocytes obtained from AdCMV.mTRH-R infected animals showed TRH-R mRNA transcripts of the expected size (see FIG. 4, lane 2). As a control, γ-actin mRNA transcripts were observed in all samples. Consistent with this observation, hepatocytes recovered from animals receiving AdCMV.mTRH-R in vivo demonstrated high levels of methyl-TRH specific binding, whereas no specific methyl-TRH binding was observed in naive animals or animals receiving the control vector AdCMV.βgal (FIG. 6). The analysis of [³H]methyl-TRH binding was identical to that described for FIG. 2. Triplicate measurements were made from three individual animals per condition. The hepatocytes derived from the AdCMV.mTRH-R infected animals demonstrated 6.1×10⁵ TRH-R receptors/cells, with a Kd of 2.51±0.32 nM. The methyl-TRH complex underwent internalization in hepatocytes from AdCMV.mTRH-R infected animals (not shown).

Finally, addition of TRH to hepatocytes from uninfected animals and animals infected in vivo with the control vector AdCMV.βgal showed no stimulation of inositol phosphate formation (FIG. 7). Measurement of inositol phosphates formation was identical to that described for FIG. 3. Triplicate measurements were made from three individual animals per condition. In contrast, TRH activated the phosphoinositide pathway in hepatocytes from animals infected 5 days previously with the AdCMV.mTRH-R vector (16.9±1.8 fold stimulation above the controls of no added methyl-TRH). Evaluation of TRH stimulation as a function of time in these cultures demonstrated an increase in the formation of inositol phosphates over a period of at least 1 hr following addition of methyl-TRH (not shown).

### EXAMPLE 4

The liver maintains blood glucose levels by secreting glucose derived from hepatocyte glycogen stores into the circulation. This process is regulated by specific cell-surface receptors activating either of two signal transduction pathways, the cAMP pathway or the phosphoinositide pathway. Both converge to activate phosphorylase B which cleaves glycogen to glucose-l-phosphate, which is then isomerized to glucose-6-phosphate by phosphoglucomutase, and finally dephosphorylated by glucose-6-phosphatase to glucose which is secreted (17).

This example discloses the modulation in levels of serum glucose by TRH in animals following in vivo transfer of the TRH-R cDNA to the liver. The vectors AdCMV.mTRH-R or AdCMV.Null (5×10⁹ pfu), or saline (0.9%) as negative control (all in 100 μl) were administered to rats as described above. Five days later, the animals were anaesthetized by methoxyflurane (Pitman-Moore, Mundelein, Ill.) inhalation and the left and right external jugular veins were cannulated. After 15 min to permit stabilization, a 15 min baseline period was started, during which serum samples for glucose levels were obtained every 5 min. At “0 time” methyl-TRH (500 μg in 100 μl) or saline (0.9%, 100 μl ) was administered in one cannula and the serum samples obtained from the other cannula every 5 min for 55 min. Serum glucose levels were determined by colormetric assay (Sigma Diagnostics, St. Louis, Mo.).

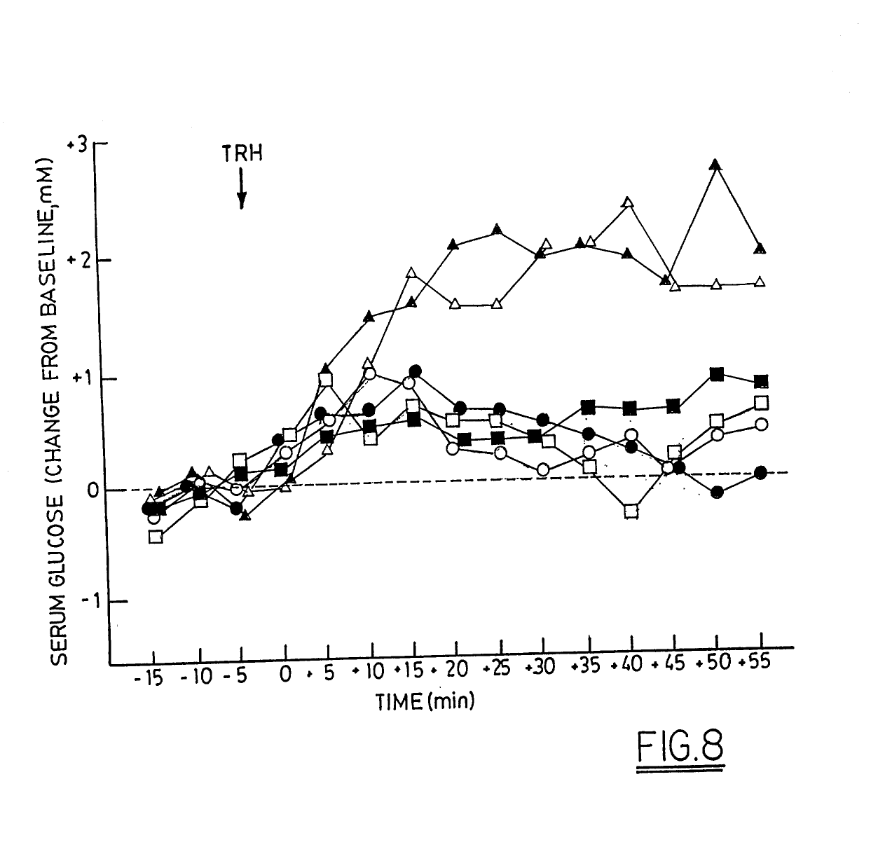

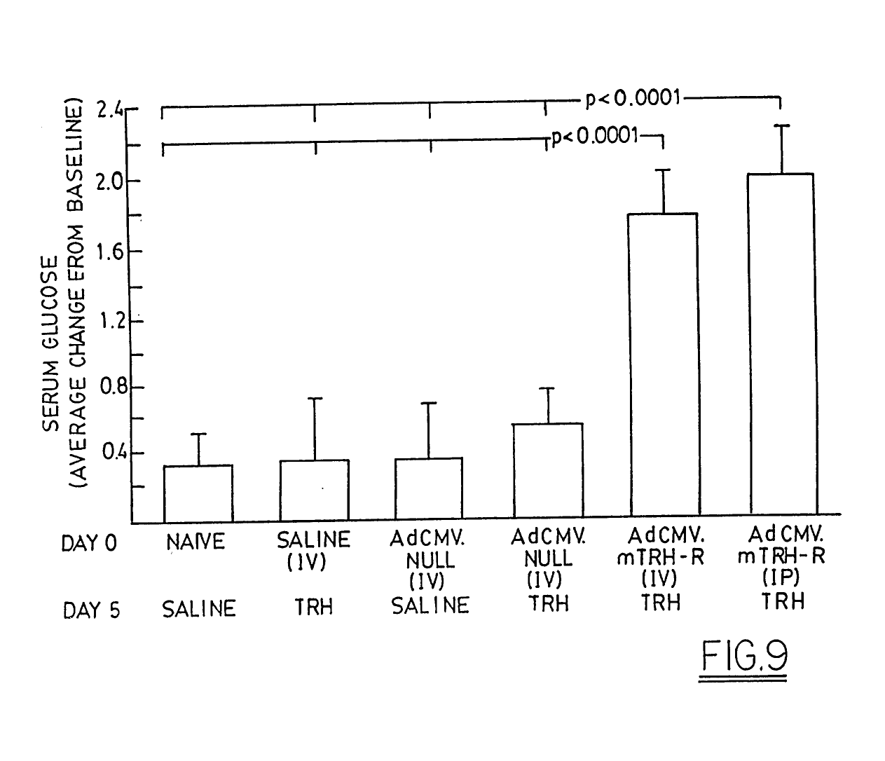

FIG. 8 illustrates the modulation of serum glucose by TRH. The baseline glucose levels were determined for each animal as an average from −15 to 0 before administration of methyl-TRH or saline. The data are presented as the absolute change from the average baseline serum glucose level (mM) using each individual animal as its own control, and averaging the values at each time point for each group of animals. AdCMV.mTRH-R (IV)/TRH=vector administered intravenously at day 0, methyl-TRH administered at day 5, n=6 animals; AdCMV.mTRH-R(IP)/TRH=vector administered via the portal vein at day 0, methyl-TRH administered at day 5, n=5; AdCMV.Null(IV)/TRH=vector administered intravenously at day 0, methyl-TRH at day 5, n=6; saline/TRH=saline administered instead of vector at day 0, methyl-TRH at day 5, n=6; and naive/saline=nothing administered at day 0, saline at day 5, n=5. The data is presented as the mean for all animals in the group at each time point. The normal range for serum glucose Sprague-Dawley rats is 2.5 6.7 mM(36). The average baseline values (mean±standard deviation) were: AdCMV.mTRH-R(IV)/TRH group 6.1±0.8 mM; AdCMV.mTRH-R(IP)/TRH group 5.6±0.5 mM; AdCMV.Null(IV)/TRH group 4.6±0.5 mM; AdCMV.Null(IV)/saline group 7.9±1.8 mM; saline (IV)/TRH group 9.2±1.4 mM; and naive/saline group 8.6±0.7 mM.

FIG. 9 illustrates the average change in serum glucose levels from baseline. The average change in serum glucose levels from baseline was determined for each time point from 15 to 55 min after administration of methyl-TRH or saline on day 5. The data are presented as mean±standard deviation. All statistical comparisons were made using the two-tailed Student's t-test.

Initial studies demonstrated that a stable blood glucose level could be established by using inhalation anesthesia and limiting environmental stimuli during the experimental studies (18). As a positive control, when [Arg⁸]vasopressin (1 82 g in 100 μl 10.9 k NaCl) was administered by the intravenous route, there was an increase in blood glucose, followed by a decrease over the 55 min period of evaluation (not shown).

Consistent with the ability of TRH to activate the phosphoinositide pathway in hepatocytes isolated from animals receiving the AdCMV.mTRH-R vector 5 days previously, and the knowledge that activation of the phosphoinositide pathway in hepatocytes initiates glycogenolysis, intravenous administration of methyl-TRH induced a significant increase in blood glucose levels in a group of animals receiving AdCMV.mTRH-R 5 days previously (FIG. 8). In the group of animals receiving saline, and 5 days later, intravenous methyl-TRH, there was a small increase in blood glucose over the baseline levels, similar to that observed in the group administered AdCMV.Null followed 5 days later by intravenous methyl-TRH, and in the naive group administered saline. However, the increase in blood glucose over baseline levels observed in each of these control groups was minimal compared to the increase in blood glucose over baseline induced by methyl-TRH in animals receiving the AdCMV.mTRH-R vector by the intravenous route 5 days previously. In these animals, the rise in blood glucose peaked 15 min after the administration of methyl-TRH, and the elevation in blood glucose levels was maintained for at least 55 min, the time at which the experiment was terminated. The increase in blood glucose over baseline from 15 to 55 min after methyl-TRH administration was 1.73±0.26 mM, an increase markedly higher than that observed in any of the controls over the same period (saline (IV)/TRH, naive/saline, AdCMV.Null (IV)/TRH and AdCMV.Null (IV)/saline, p<0.0001 all comparisons) (FIG. 9).

Direct support for the concept that the liver was responsible for the rise in blood glucose came from administration of the AdCMV.mTRH-R vector to rats via the portal vein [a route of administration of an adenovirus vector to rats known to result in limitation of vector gene expression only in hepatocytes (19,20)], followed 5 days later by the administration of intravenous methyl-TRH. In these animals, the observed rise in blood glucose was superimposable upon that observed in animals receiving the AdCMV.mTRH-R vector intravenously followed 5 days later by administration of methyl-TRH (AdCMV.mTRH-R administered via portal vein compared to AdCMV.mTRH-R administered intravenously averaged 15 to 55 min after methyl-TRH, p=0.09; portal vein administration of AdCMV.mTRH-R followed by methyl-TRH compared to control groups, p<0.0001 all comparisons) (FIGS. 8 and 9).

### EXAMPLE 5

The present invention supports the concept that ectopic expression of a non-endogenous naturally occurring receptor can be used to control differentiated functions of cells in vivo, using the natural ligand for the receptor to activate the receptor, and in turn, activate a specific signal transduction pathway in the cell, thus triggering a cell-specific response. Put into the context of the availability of vectors that can transfer genes in vivo to most organs, the strategy can be used as an alternative means to modulate specific differentiated functions of cells using naturally occurring ligands or receptor-specific drugs and their corresponding receptors not normally relevant to that cell type. The strategy of expressing natural receptors as described herein adds to a growing list of strategies to modify receptor number and/or function, including overexpression of natural receptors in their natural location (21) and “designer” receptors that respond to artificial ligands (22) as means of triggering specific signal transduction pathways to activate specific differentiated functions of cells, and the transfer of normal receptors to their normal location to compensate for mutations in the natural receptor (23,24).

Clinical applications of this strategy include activation of differentiated functions that are impotent and/or dysfunctional secondary to inherited or acquired abnormalities associated with signalling molecules or their specific receptors. In addition to deficiencies in signalling secondary to mutations in genes coding for hormones or receptors (1,6,25,26), such a strategy could be used to bypass acquired ligand deficiency states associated with antibodies directed against the ligand, such as observed in individuals with diabetes receiving insulin of human and non-human origin, polyclonal anti-insulin antibodies in individuals with various autoimmune disorders and antithyroxine antibodies in patients with immune thyroid disease and plasma cell dyscrasias (1,6,7).In addition, there are a group of acquired disorders in which receptor-specific autoantibodies intercept the ability of extracellular regulatory molecules to interact with their specific receptors, including the autoantibodies against β1 -adrenergic receptors implicated in the pathogenesis of idiopathic dilated cardiomyopathy and the cardiomyopathy associated with Chagas' disease, autoantibodies against β2-adrenergic receptors linked to the pathogenesis of some forms of asthma, antibodies to the insulin receptor associated with the diabetes found and type B insulin resistance observed in individuals with acanthosis nigricans and ataxia telangiectasia, autoantibodies to the acetylcholine receptor in myasthenia gravis and to the glutamate receptor in Rasmussen's encephalitis (1,6,7,27,28).Finally, this strategy may also be applied to the treatment of diseases for which regulation of a physiologic function in a healthy organ would counterbalance the effects of a disease process. An example of this is suggested by the previous examples, in which blood glucose was elevated by TRH after ectopic expression of TRH-Rs in liver. This approach could be used in a patient with severe hypoglycemia who harbors an inoperable insulin-secreting tumor (insulinoma) in order to maintain normoglycemia over an extended period of time (29). Not only could this be achieved by triggering the phosphoinositide pathway (e.g., TRH and TRH-Rs expressed in liver), but perhaps more effectively, by expressing in the liver a non-endogenous receptor that signals via the cAMP transduction system, since glycogenolysis in human liver is more effectively regulated by this pathway than by the phosphoinositide pathway, and for which oral non-peptide agonist drugs are available for activation.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

### LIST OF REFERENCES CITED

1. S. Gammeltoft and R. C. Kahn, Endocrinology, L. J. DeGroot, et al., Eds., W.B. Saunders Company, Philadelphia (1995), p. 17.

2. M. C. Gershengorn and J. H. Perlman, Endocrinology, L. J. DeGroot, et al., Eds., W.B. Saunders Company, Philadelphia (1995), p. 66.

3. F. Habener, Endocrinology, L. J. DeGroot, et al., Eds., W. B. Saunders Company, Philadelphia (1995), p. 77.

4. D. A. Walsh and S. M. Van Patten, FASEB J 8:1227 (1994).

5. J. H. Exton, Diabetes Metab Rev 3:163 (1987).

6. J. R. Raymond, Am J Physiol 266:F163 (1994).

7. T. J. Wilkin, N Engl JMed 323:1318 (1990)

8. Y. Tsunoda, Biochim Biophys Acta 1154:105 (1993).

9. L. Birnbaumer, et al., Crit Rev Biochem Mol Biol 25:225 (1990).

10. M. C. Gershengom, et al., J Biol Chem 269:6779 (1994).

11. F. R. Bringhurst et al., Endocrinology 132:2090 (1993).

12. K. J. Kaneko, et al., Biochemistry 32:8348 (1993).

13. M. C. Gershengorn, Recent Prog Horm Res 48:341 (1993).

14. M. F. Scanlon and R. Hall, Endocrinology, L. J. DeGroot, et al., Eds., W. B. Saunders Company, Philadelphia (1995), p. 192.

15. W. Vale, et al., Endocrinology 89:1485 (1971).

16. J. Herz and R. D. Gerard, Proc Natl Acad Sci 90:2812 (1993).

17. S. J. Pilkis and D. K. Granner, Annu Rev Physiol 54:885 (1992).

18. A. A. Young, et al., Am J Physiol 264:E943 (1993)

19. H. A. Jaffe et al., Nat Genet 1:372 (1992).

20. Q. Li, et al., Hum Gene Ther 4:403 (1993).

21. C. A. Milano, et al., Science 264:582 (1994).

22. D. M. Spencer, et al., Science 262:1019 (1993).

23. M. Grossman, et al., Nat Genet 6:335 (1994).

24. K. F. Kozarsky, et al., J Biol Chem 269:13695 (1994).

25. R. J. Lefkowitz and R. T. Premont, J Clin Invest 92:2089 (1993).

26. A. M. Spiegel, et al., J Clin Invest 92:1119 (1993).

27. S. W. Rogers, et al., Science 265:648 (1994).

28. Y. Naparstek and P. H. Plotz, Annu Rev Immunol 11:79 (1993).

29. J. A. Norton, Curr Probl Surg 31:77 (1994).

30. E. Falck-Pedersen, et al., Mol Pharmacol 45:684 (1994).

31. J. Hersh, et al., Gene Therapy 2:124 (1995).

32. M. A. Rosenfeld, et al., Science 252:431 (1991)

33. M. A. Rosenfeld, et al., Cell 68:143 (1992).

34. M. C. Gershengorn, J Clin Invest 62:937 (1978).

35. A. Imai and M. C. Gershengorn, Methods Enzymol 141:100 (1987).

36. B. M. Mitruka and H. M. Rawnsley, Clinical Biochemical and Hematological Reference Values in Normal Experimental Animals, Masson, N.Y. (1977), p. 117.

37. Barritt, G. J. Communication Within Animal Cells. Oxford, UK: Oxford Science Publications, 1992.

38. Hardie, D. G. Biochemical Messengers: Hormones, Neurotransmitters and Growth Factors. London: Chapman and Hall. 1990.

39. Molecular Biology of Signal Transduction. Cold Spring Harbor Symposium Quant. Biol., Vol. 53. 1988.

40. Morgan, N. G. Cell Signalling. Milton Kaynes, UK: Open University Press, 1989.

41. Kahn, C. R., J Cell Biol 70:261-286 (1976).

42. Snyder, S. H., Sci Am 253(4):132-140 (1985).

43. Straub et al., Proc Natl Acad Sci USA, 87:9514-9518 (1990).

44. Huang et al., Nucleic Acids Res, 18:937947 (1990).

45. Tantravahi et al., Mol Cell Biol 13:578587 (1993).

46. Graham et al., J Gen Virol 36:59-72 (1977).

47. Mannino, R. J. and S. Gould-Fogerite, BioTechniques 6:682-690 (1988).

48. Shigekawa, K. and W. J. Dower, BioTechniques 6:742-751 (1988).

49. Capecchi, M., Cell 22:479-488 (1980).

50. Klein, T. M., et al., Nature 327:70-73 (1987).

51. Miller, L. K., Bioessays 11:91-95 (1989).

52. Cepko, C., Neuron 1:345-353 (1988).

53. Eglitis, M. A. and W. F. Anderson, BioTechniques 6:608-614 (1988).

54. Berkner, K. L., BioTechniques 6:616-629 (1988).

55. Friedmann, T., Science 244:1275-1281 (1989).

56. Verma, I. M., Sci Am 262:68-84 (1990).

57. Corey, C. A., et al., Blood 75:337-343 (1990)

58. Palmer, T. D., et al., Proc Natl Acad Sci USA 84:1055-1059 (1987).

59. Anderson, K. D., et al., Somat Cell Mol Genet 15:215-217 (1989).

60. Nabel, E. G., et al., Science 244:13421344 (1989).

61. Nabel, E. G., et al., Science 249:12851288 (1990).

62. Anderson, W. F., Science 256:808-813 (1992).

63. Mulligan, R. C., Science 260:926-932 (1993).

What is claimed is:

1. A recombinant in vivo mammalian cell comprising an endogenous signal transduction pathway that is triggered by the interaction of an endogenous cell signaling receptor with a first ligand,
wherein DNA encoding a non-endogenous cell signaling receptor that interacts with a second ligand to trigger the endogenous signal transduction pathway has been introduced into the in vivo mammalian cell,
wherein the non-endogenous cell signaling receptor is different from the endogenous cell signaling receptor,
wherein the first and second ligands are different, and
wherein the non-endogenous cell signaling receptor is controllably activated, thereby controllably activating the endogenous signal transduction pathway so as to regulate a cellular function controlled by said endogenous signal transduction pathway.

2. The recombinant in vivo mammalian cell of claim 1, wherein said cell is a cell of an organ.

3. The recombinant in vivo mammalian cell of claim 2, wherein said organ is a liver and said cell is a hepatocyte.

4. The recombinant in vivo mammalian cell of claim 1, wherein said non-endogenous cell signaling receptor is on the surface of the cell.

5. The recombinant in vivo mammalian cell of claim 4, wherein said non-endogenous cell signaling receptor is a guanine-nucleotide binding protein linked receptor.

6. The recombinant in vivo mammalian cell of claim 5, wherein said guanine-nucleotide binding protein linked receptor is selected from the group consisting of alpha-adrenersic, receptors, beta-adrenergic receptors, dopaminergic receptors, serotonergic receptors, muscarinic cholinergic receptors, and peptidergic receptors.

8. The recombinant in vivo mammalian cell of claim 5, wherein said guanine-nucleotide binding protein linked receptor is thyrotropin releasing hormone receptor.

8. The recombinant in vivo mammalian cell of claim 1, wherein the signal transduction pathway is selected from the group consisting of an adenylate cyclase pathway, a guanylate cyclase pathway, a phosphoinositol turnover pathway, a tyrosine kinase pathway, an ion channel pathway, and a calcium ion pathway.

9. The recombinant in vivo mammalian cell of claim 1, wherein said cell function is glycogenolysis.

10. The recombinant in vivo mammalian cell of claim 1, wherein said cell function is selected from the group consisting of lipolysis, gluconeogenesis, ketogenesis, ion permeability, renin production, muscle contraction, protein phosphorylation, thyroid hormone synthesis, cortisol secretion, progesterone secretion, bone resorption, water resorption, triglyceride breakdown, amylase secretion, histamine secretion, and platelet aggregation.

11. A method of ectopically expressing a non-endogenous receptor in a cell in vivo, said method comprising:
selecting an in vivo mammalian cell comprising an endogenous signal transduction pathway that is triggered by the interaction of an endogenous cell signaling receptor with a first ligand;
introducing into the in vivo mammalian cell DNA encoding a non-endogenous cell signaling receptor that interacts with a second ligand to trigger the endogenous signal transduction pathway, wherein the non-endogenous cell signaling receptor is different from the endogenous cell signaling receptor, and the first and second ligands are different; and
ectopically expressing the DNA encoding the non-endogenous cell signaling receptor in the cell.

12. The method of claim 11, wherein the introduction of DNA encoding a non-endogenous cell signaling receptor into the mammalian cell is viral mediated.

13. The method of claim 12, wherein the introduction of DNA encoding a non-endogenous cell signaling receptor into the mammalian cell is adenoviral mediated.

14. The method of claim 11, wherein said cell is a cell of an organ.

15. The method of claim 14, wherein said organ is a liver and said cell is a hepatocyte.

16. The method of claim 11, wherein said non-endogenous cell signaling receptor is on the surface of the cell.

17. The method of claim 37, wherein said non-endogenous cell signaling receptor is a guanine-nucleotide binding protein linked receptor.

18. The method of claim 17, wherein said guanine-nucleotide binding protein linked receptor is selected from the group consisting of alpha-adrenergic receptors, beta-adrenergic receptors, dopaminergic receptors, serotonergic receptors, muscarinic cholinergic receptors, and peptidergic receptors.

19. The method of claim 17, wherein said guanine-nucleotide binding protein linked receptor is a thyrotropin releasing hormone receptor.

20. The method of claim 16, wherein said non-endogenous cell signaling receptor is linked to an enzyme.

21. The method of claim 20, wherein said non-endogenous cell signaling receptor is selected from the group consisting of transmembrane guanylyl cyclase receptors, receptor tyrosine phosphatases, transmembrane receptor serine/threonine kinases, receptor tyrosine kinases, and tyrosine-kinase-associated receptors.

22. The method of claim 11, wherein said signal transduction pathway is selected from the group consisting of an adenylate cyclase pathway, a guanylate cyclase pathway, a phosphoinositol turnover pathway, a tyrosine kinase pathway, an ion channel pathway, and a calcium ion pathway.

23. The method of claim 11, wherein said cell function is glycogenolysis.

24. The method of claim 11, wherein said cell function is selected from the group consisting of lipolysis, gluconeogenesis, ketogenesis, ion permeability, renin production, muscle contraction, protein phosphorylation, thyroid hormone synthesis, cortisol secretion, progesterone secretion, bone resorption, water resorption, triglyceride breakdown, amylase secretion, histamine secretion, and platelet aggregation.

25. A method of regulating a mammalian cell function in vivo, said method comprising:
selecting a mammalian cell comprising an endogenous signal transduction pathway that is triggered by the interaction of an endogenous cell signaling receptor with a first ligand;
introducing into the mammalian cell DNA encoding a non-endogenous cell signaling receptor that interacts with a second ligand to trigger the endogenous signal transduction pathway, wherein the non-endogenous cell signaling receptor is different from the endogenous cell signaling receptor, and the first and second ligands are different;
ectopically expressing the non-endogenous cell signaling receptor in the mammalian cell; and
controllably exposing the mammalian cell in vivo to the second ligand that activates the non-endogenous cell signaling receptor, whereupon activation of the non-endogenous cell signaling receptor triggers the endogenous signal transduction pathway so as to regulate a cellular function controlled by said endogenous signal transduction pathway.

26. The method of claim 25, wherein the introduction of DNA encoding a non-endogenous cell signaling receptor into the mammalian cell is viral mediated.

27. The method of claim 26, wherein the introduction of DNA encoding a non-endogenous cell signaling receptor into the mammalian cell is adenoviral mediated.

28. The method of claim 25, wherein said cell is a cell of an organ.

29. The method of claim 28, wherein said organ is a liver and said cell is a hepatocyte.

30. The method of claim 25, wherein said non-endogenous cell signaling receptor is on the surface of the cell.

31. The method of claim 30, wherein said non-endogenous cell signaling receptor is a guanine-nucleotide binding protein linked receptor.

32. The method of claim 31, wherein said guanine-nucleotide binding protein linked receptor is selected from the group consisting of alpha-adrenergic receptors, beta-adrenergic receptors, dopaminergic receptors, serotonergic receptors, muscarinic cholinergic receptors, and peptidergic receptors.

33. The method of claim 31, wherein said guanine-nucleotide binding protein linked receptor is a thyrotropin releasing hormone receptor.

34. The method of claim 30, wherein said non-endogenous cell signaling receptor is linked to an enzyme.

35. The method of claim 34, wherein said non-endogenous cell signaling receptor is selected from the group consisting of transmembrane guanylyl cyclase receptors, receptor tyrosine phosphatases, transmembrane receptor serine/threonine kinases, receptor tyrosine kinases, and tyrosine-kinase-associated receptors.

36. The method of claim 25, wherein said signal transduction pathway is selected from the group consisting of an adenylate cyclase pathway, a guanylate cyclase pathway, a phosphoinositol turnover pathway, a tyrosine kinase pathway, an ion channel pathway, and a calcium ion pathway.

37. The method of claim 25, wherein said cell function is glycogenolysis.

38. The method of claim 25, wherein said cell function is selected from the group consisting of lipolysis, gluconeogenesis, ketogenesis, ion permeability, renin production, muscle contraction, protein phosphorylation, thyroid hormone synthesis, cortisol secretion, progesterone secretion, bone resorption, water resorption, triglyceride breakdown, amylase secretion, histamine secretion, and platelet aggregation.

39. The method of claim 25, wherein said second ligand comprises a drug.

40. The recombinant in vivo mammalian cell of claim 1, wherein said endogenous cell signaling receptor is mutated or absent from the mammalian cell.

41. The recombinant in vivo mammalian cell of claim 2, wherein said organ is a heart and said cell is a cardiac muscle cell.

42. The method of claim 14, wherein said organ is a heart and said cell is a cardiac muscle cell.

43. The method of claim 28, wherein said organ is a heart and said cell is a cardiac muscle cell.

44. The recombinant in vivo mammalian cell of claim 3, wherein said non-endogenous cell signaling receptor is a guanine-nucleotide binding protein linked receptor.

45. The recombinant in vivo mammalian cell of claim 44, wherein said guanine-nucleotide binding protein linked receptor is thyrotropin releasing hormone receptor.

46. The method of claim 45, wherein the introduction of DNA encoding a non-endogenous cell signaling receptor into the in vivo mammalian cell is adenoviral mediated.

47. The method of claim 15, wherein the introduction of DNA encoding a non-endogenous cell signaling receptor into the in vivo mammalian cell is adenoviral mediated.

48. The method of claim 47, wherein said non-endogenous cell signaling receptor is a guanine-nucleotide binding protein linked receptor.

49. The method of claim 48, wherein said guanine-nucleotide binding protein linked receptor is a thyrotropin releasing hormone receptor.

50. The method of claim 29, wherein the introduction of DNA encoding a non-endogenous cell signaling receptor into the mammalian cell is adenoviral mediated.

51. The method of claim 50, wherein said non-endogenous cell signaling receptor is a guanine-nucleotide binding protein linked receptor.

52. The method of claim 51, wherein said guanine-nucleotide binding protein linked receptor is a thyrotropin releasing hormone receptor.
